(19) United States
(12) Patent Application Publication
(10) Pub. No.: US 2010/0234934 A1
(43) Pub. Date: Sep. 16, 2010

(54) Balloon Deployable Coronary Stent

(75) Inventor: ERIC YU, San Francisco, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/400,875

(22) Filed: Mar. 10, 2009

(51) Int. Cl. A61F 2/06

(52) U.S. Cl. 623/1.12; 623/1.11; 29/525.01; 623/1.42; 623/1.43

(57) ABSTRACT

A system for treating a vascular condition includes a delivery catheter, a balloon disposed at a distal end of the delivery catheter, a stent having a stent framework, the stent disposed on an outer surface of the balloon; and at least one restraining filament attached to an outer surface of the stent. a method of treating a vascular condition includes delivering a self-expanding stent including a restraining filament to a treatment site via a balloon catheter, the stent disposed over the balloon; inflating the balloon to fracture at least one fracture point disposed along the length of the restraining filament; expanding the self-expanding stent at the treatment site; contacting an outer surface of the self-expanding stent with a vessel wall at the treatment site; and trapping the fractured restraining filament between at least a portion of an outer surface of the stent and the vessel wall.

### FIELD OF THE INVENTION

This invention relates generally to biomedical stents. More specifically, the invention relates to a balloon deployable drug coated coronary stent having increased flexibility and decreased crossing profile.

### BACKGROUND OF THE INVENTION

Recent advances in medical procedures such as angioplasty continue to increase the use of endovascular stents in a variety of treatments for unblocking body lumens and restoring their function. Stents help reduce the probability and degree of vessel blockage from restenosis. Commonly used stents are balloon expandable or self-expanding. Balloon expandable stents are deployed by mounting the stent on a balloon portion of a balloon catheter, positioning the stent in a body lumen, and expanding the stent by inflating the balloon. The balloon is then deflated and removed, leaving the stent in place. Self-expanding stents are manufactured from material that is set to assume an expanded configuration when released from a delivery catheter at a treatment site. Self expanding stents are encased within a sheath that is removed in order to release the stent from a delivery catheter.

As may be expected, stents that are suitable for one application may not be suitable for another, different application due to location of the treatment site, type of stent and the material used for manufacturing the stent. For example, balloon expandable stents composed of rigid stainless steel or cobalt chromium alloy may not be suitable for applications where a more flexible stent is required. Also, a self-expanding stent made of nitinol, though flexible, may not be suitable for areas where crossing profile is an issue due to the need for a restraining sheath that increases a delivery catheter's crossing profile.

Medical research indicates an increased effectiveness of vascular stents when stents are coated with pharmaceutical drugs that help prevent or treat medical conditions such as restenosis and thrombosis. Stent coatings provide localized therapeutic pharmacological agents and treatment of a vessel at the site being supported by the stent to deliver patent effects at the site where they are most needed. The localized levels of the medications can be elevated, and therefore potentially more effective than orally or intravenously delivered drugs. Furthermore, drugs released from tailored stent coatings can have controlled, timed-release qualities, eluting their bioactive agents over hours, weeks or even months.

However, one drawback of self-expanding stents having a therapeutic coating is that the sheath restraining the stent may cause loss of coating due to constant contact between the inner surface of the sheath and the coated stent surface during the packaging, delivery and deployment processes.

Accordingly, what is needed is an improved self-expanding stent with a therapeutic coating that is flexible, has a reduced crossing profile, and overcomes other deficiencies and limitations described above.

### SUMMARY OF THE INVENTION

One aspect of the invention provides a system for treating a vascular condition. The system includes a delivery catheter, a balloon disposed at a distal end of the delivery catheter, a stent having a stent framework, the stent disposed on an outer surface of the balloon; and at least one restraining filament attached to an outer surface of the stent.

Another aspect of the invention provides a method of manufacturing a vascular treatment device. The method includes providing a stent, the stent having a stent framework; securing a first end of a restraining filament to the stent framework; placing the stent over a balloon disposed at a distal end of a delivery catheter; compressing the stent onto an outer surface of the balloon; wrapping the restraining filament around the stent; and securing a second end of the restraining filament to the stent framework.

Another aspect of the invention provides a method of treating a vascular condition. The method includes delivering a self-expanding stent including a restraining filament to a treatment site via a balloon catheter, the stent disposed over the balloon; inflating the balloon to fracture at least one fracture point disposed along the length of the restraining filament; expanding the self-expanding stent at the treatment site; contacting an outer surface of the self-expanding stent with a vessel wall at the treatment site; and trapping the fractured restraining filament between at least a portion of an outer surface of the stent and the vessel wall.

The present invention is illustrated by the accompanying drawings of various embodiments and the detailed description given below. The drawings should not be taken to limit the invention to the specific embodiments, but are for explanation and understanding. The detailed description and drawings are merely illustrative of the invention rather than limiting, the scope of the invention being defined by the appended claims and equivalents thereof. The foregoing aspects and other attendant advantages of the present invention will become more readily appreciated by the detailed description taken in conjunction with the accompanying drawings.

### BRIEF DESCRIPTION OF THE DRAWINGS

Various embodiments of the present invention are illustrated by the accompanying figures, wherein:

FIG. 1 is an illustration of a system for treating a vascular condition including a self expanding stent coupled to a catheter, in accordance with one embodiment of the current invention;

FIG. 2 is a detailed view of one portion of the stent of FIG. 1, in accordance with one embodiment of the current invention;

FIG. 3 is a detailed portion of a restraining filament, in accordance with one embodiment of the current invention;

FIG. 4 is a detailed view of one portion of one embodiment of a stent framework, in accordance with the current invention;

FIG. 5 is a flow diagram of a method of manufacturing a self expanding stent with a restraining filament, in accordance with one embodiment of the current invention; and

FIG. 6 is a flow diagram of a method for treating a vascular condition, in accordance with one embodiment of the current invention.

### DETAILED DESCRIPTION OF THE PRESENTLY PREFERRED EMBODIMENTS

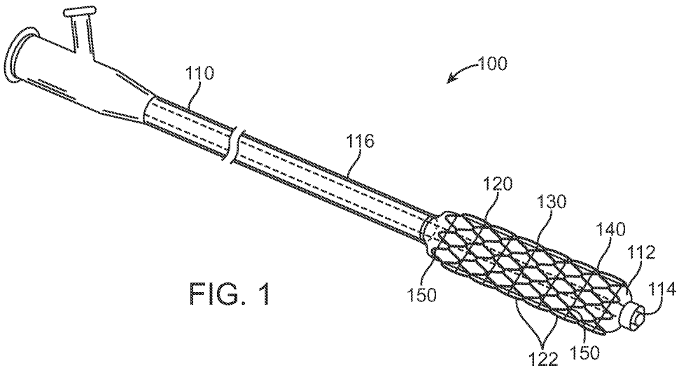

FIG. 1 shows an illustration of a system 100 for treating a vascular condition. System 100 comprises a self-expanding drug coated stent coupled to a balloon catheter, in accordance with one embodiment of the present invention. System 100 includes a self-expanding drug coated stent 120 coupled to a delivery catheter 110. Stent 120 includes a self-expanding stent framework 130. Stent framework 130 comprises a metallic base having shape memory properties such as, for example, nitinol. In one embodiment, stent 120 comprises a plurality of stent segments 122 placed end-to end. In another embodiment, stent 120 is manufactured by cutting a sheet or tube of material into stent 120 or stent segments and then attaching the stent segments to form stent 120. Those with ordinary skill in the art will recognize that the length of stent 120 or the number of stent segments comprising stent 120 may vary depending on a particular application and the particular characteristics of the treatment site.

Stent 120 may also include a drug coating 140 disposed on the stent framework. In one embodiment, drug coating 140 includes at least one therapeutic agent or drug. Throughout, the terms “therapeutic agent” and “drug” are used interchangeably and refer to any agent having a therapeutic effect. A therapeutic agent is capable of producing a beneficial effect against one or more conditions including coronary restenosis, cardiovascular restenosis, angiographic restenosis, arteriosclerosis, hyperplasia, and other diseases or conditions. The therapeutic agent may be, for example, anticoagulants, antiinflammatories, fibrinolytics, antiproliferatives, antibiotics, therapeutic proteins, recombinant DNA products, bioactive agents, diagnostic agents, radioactive isotopes, and radiopaque substances.

In one embodiment, drug coating 140 includes at least one drug-polymer layer. In another embodiment, drug coating 140 includes a plurality of thin drug-polymer layers. The drug-polymer layers include at least one therapeutic agent and at least one polymer. Drug coating 140 may include at least one barrier layer positioned between one or more thin drug and/or drug-polymer layers. The constituents of coated stent 120 are selected to provide a pre-determined drug-release profile. Drug coating 140 may comprise one or more therapeutic agents dispersed within drug layers, drug-polymer layers and/or barrier layers on coated stent 120, which are eluted from coated stent 120 with controlled time delivery after deployment of stent 120 into the body.

Catheter 110 further includes a balloon 112, disposed at a distal end. Balloon 112 is in fluid communication with an inflation fluid source (not shown) via inflation lumen 116 of catheter 110. Balloon 112 is inflated by pressurizing a fluid such as a dilute contrast fluid that fills lumen 116 inside catheter 110 and balloon 112. Stent 120 is disposed upon an outer surface of balloon 112.

Catheter 110 includes at least one restraining filament 150. Restraining filament 150 comprises a thin filament or thread composed of a biodegradable material such as, for example, polymers or copolymers of lactide, glycolide, trimethylene carbonate and poly(ethylene glycol). FIG. 3 illustrates a portion of one embodiment of a restraining filament 150. Restraining filament 150 includes at least one fracture point 156 along the length of restraining filament 150. Fracture point 156 can be formed by scoring filament 150 or by controlled thinning of filament 150. Controlling fracture points will prevent random thread fracture at securement regions and will ensure that restraining filament 150 remains in the stent after deployment. The location and number of fracture points 156 within a restraining filament are determined so that when in use the restraining filament is not released from stent 120. Configuring the fracture points in this manner is done so that once the stent is deployed, the restraining filament remains attached to the stent to biodegrade or otherwise be absorbed into the body.

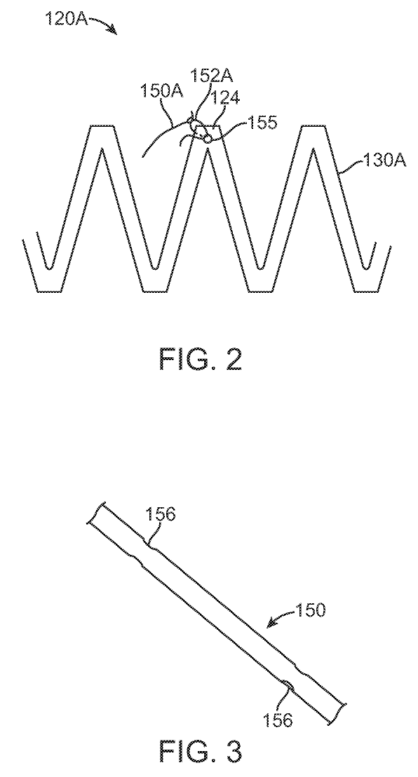

Restraining filament 150 is attached to stent 120. FIG. 2 illustrates a detailed portion of stent 120A showing one embodiment for attaching restraining filament 150A to stent 120A. In one embodiment, stent 120A includes at least one filament attachment 155 defined by an opening at or near a crown portion 124 of stent framework 130A. A first end 152A of restraining filament 150A is attached to stent 120A by threading first end 152A through filament attachment 155 and securing first end 152A to stent framework 130 by forming a knot in restraining filament 150A.

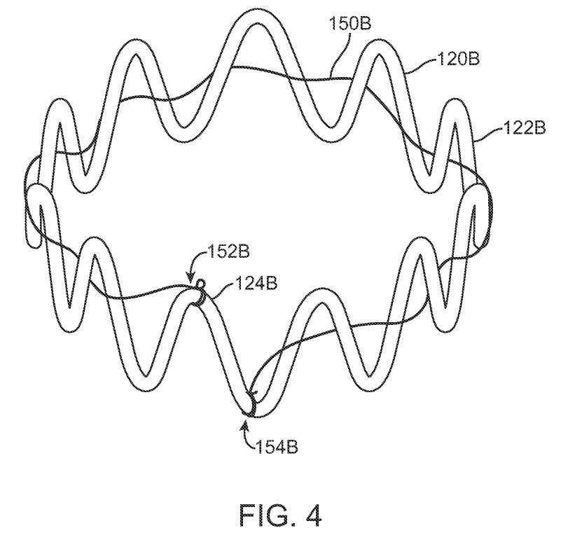

Restraining filament 150 may be attached to stent 120 by tying a knot, adhesive or any other means suitable for securing an end of restraining filament to a portion of the stent framework. Referring to FIG. 4, restraining filament 150B is attached to stent 120B by wrapping a portion of first end 152B around a crown portion 124B and securing end 152B by tying a knot. In another embodiment, restraining filament 150 may be attached to stent 120 by adhesive, or welding or any other suitable securing means.

Referring to FIG. 1, in one embodiment, restraining filament 150 is wrapped around self-expanding stent 120 after stent 120 is disposed over balloon 112. In this embodiment, after stent 120 is compressed or crimped onto balloon 112, restraining filament 150 is wrapped around stent 120 from the first end attachment to an end opposite the first end attachment. For example, where the first end attachment is at a distal end of stent 120, restraining filament is wrapped around the outer surface of stent 120 from the distal end towards the proximal end. Restraining filament 150 is wrapped around any portion of stent 120. In one embodiment, Restraining filament 150 is wrapped around each stent segment 122 along the length of stent 120. In another embodiment, restraining filament 150 is wrapped around stent 120 in a serpentine fashion contacting a portion of each stent segment. Restraining filament 150 is wound around stent 120 to ensure a fully crimped state and to restrain the stent in a non-expanded state. Restraining filament 150 may be wrapped around stent 120 to further compress stent 120 onto balloon 112, thereby providing a secondary compression to the stent. The secondary compression further decreases the crossing profile of stent delivery balloon catheter 110.

Referring to FIG. 4, in one embodiment, each stent segment 122B includes a restraining filament 150B. In this embodiment, a first end 152B of a restraining filament 150B is attached to a crown 124B. Once first end 152B is attached, a second end 154B is wrapped around stent segment 122B. Restraining filament 150B may be woven or threaded around and through the stent framework of stent segment 122B. In one embodiment, restraining filament 150B may be woven through openings defined within the stent framework similar to or the same as opening 155 illustrated in FIG. 2. In one embodiment stent segments 122 further include a plurality of openings 155 disposed throughout the stent framework. These openings may be disposed in the crown portions and the straight strut portions between each crown portion. Second end 154B is secured to the stent framework adjacent end 152B. Second end 154B may be secured to stent segment 122B by wrapping end 154B around a stent strut or may be threaded through an opening as described above. Second end 154B may also be secured by adhesive, welding or any other suitable securement means. In one embodiment, stent 120B, having a plurality of restraining filaments 150B wrapped around each of the stent segments 122B, is placed over balloon 112 and compressed or crimped. Stent 120B is then restrained from expanding by pulling on each of the restraining filaments 150B and securing each second end to maintain the compression. In one embodiment, stent 120B may be and further compressed by pulling on the restraining filaments. Second end 154B is secured to the stent framework as described above.

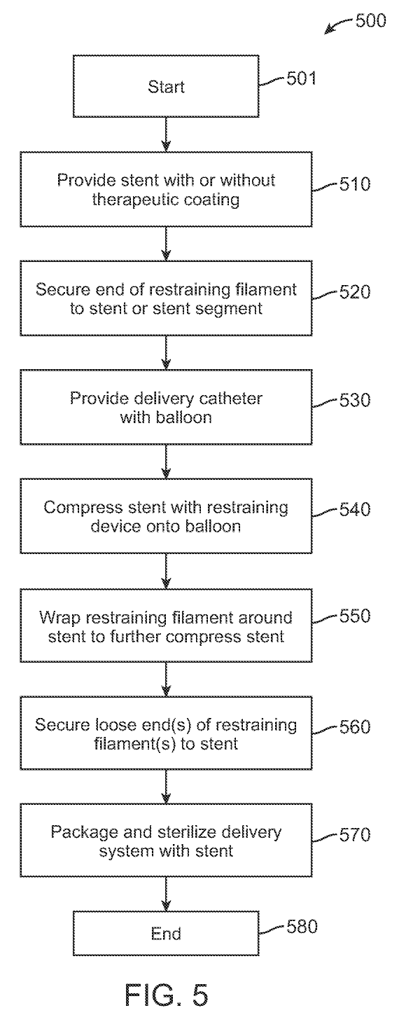

FIG. 5 illustrates a flow diagram of a method 500 of manufacturing one embodiment of system 100 described above with reference to FIGS. 1 to 4. Method 500 begins at 501. At Block 510 a stent is provided such as stent 120 having stent framework 130. A therapeutic coating 140 is applied to clean outer surface of stent 120. Therapeutic coating 140 may be a drug coating or a drug-polymer coating as described above. Therapeutic coating 140 may be a single layer coating or a multi-layer coating. Additionally, stent 120 may also include a primer coat and/or a cap coat as are known in the art. The primer coat, therapeutic coating and cap coat may be applied to the stent framework by dipping, spraying, painting, brushing, or other suitable methods. Those with skill in the art will appreciate that method 500 may include a stent that does not have a therapeutic coating.

At Block 520, a first end of at least one restraining filament 150 is secured to at least one stent segment 122 of stent 120. In one embodiment, each stent segment 122B comprising stent 120 includes a restraining filament 150 such as restraining filaments 150B described above and illustrated in FIG. 4.

Next, a balloon delivery catheter 110 is provided and the coated stent 120, having the at least one restraining filament 150, is disposed over inflatable balloon 112 (Block 530). At Block 540, coated stent 120 is compressed or crimped onto the outer surface of balloon 112. Restraining filament 150, 150A, 150B is then wrapped around stent 120 to restrain the sent from expanding and to further compress stent 120 onto balloon 112 (Block 550). Second end(s) 154 of restraining filament(s) 150 is/are secured to stent 120 (Block 560). The catheter with the stent may be placed in a catheter package and sterilized prior to shipping and storing. Before clinical use, the stent is sterilized by any appropriate or medically conventional means (Block 570). Method 500 ends at 580.

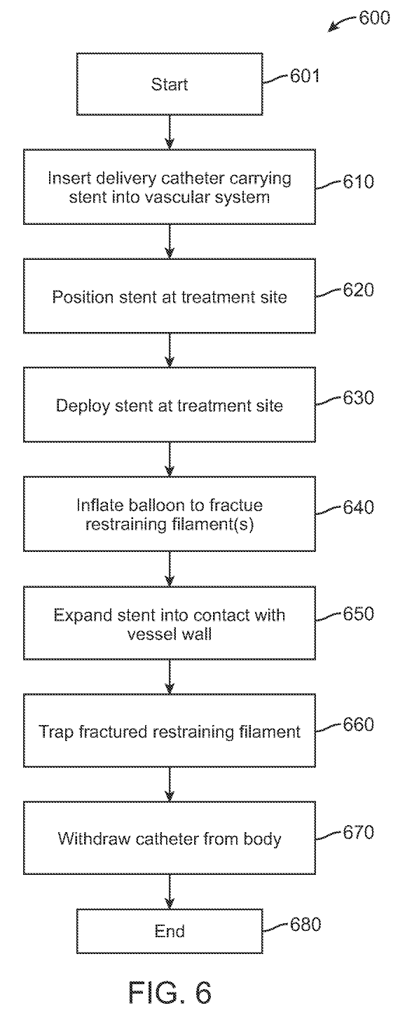

FIG. 6 illustrates a flow diagram of a method for treating a vascular condition, in accordance with one embodiment of the present invention at 600. Vascular condition treatment method 600 includes steps to insert a stent 120 having at least one restraining filament within a vessel of a body, such as, for example the coronary artery. In one embodiment, stent 120 is a coated stent and method 600 includes eluting at least one therapeutic agent from the drug coated stent into the body. Method 600 begins at 601. A delivery catheter having a stent secured with at least one restraining filament is provided.

When ready for deployment, a distal end of catheter 110 having a balloon and a stent disposed on the balloon is inserted into a vessel of the body (Block 610). The stent is inserted typically in a controlled environment such as a catheter lab or hospital. A delivery catheter is typically inserted through a small incision of the leg and into the femoral artery, and directed through the vascular system to a desired place in the patient's vascular system. At Block 620, the stent is positioned at a treatment site. Guide wires threaded through an inner lumen of the delivery catheter may assist in positioning and orienting the stent. The position of the drug-polymer coated stent may be monitored, for example, with a fluoroscopic imaging system or an x-ray viewing system in conjunction with radiopaque markers on the coated stent, radiopaque markers on the delivery catheter, or contrast fluid injected into an inner lumen of the delivery catheter and into an inflatable catheter balloon that is coupled to the stent.

Stent 120 is deployed at Block 630. Stent 120 is deployed by first expanding balloon 112 with sufficient fluid pressure to fracture restraining filament at fracture point 156 (Block 640). Once the at least one restraining filament is fractured, balloon 112 may be deflated. Fracture of restraining filament 150 allows stent 120 to self-expand into contact with the vessel wall (Block 650). Expansion of stent 120 into contact with the vessel wall traps the biodegradable filament between the stent and the vessel wall and prevents the migration of restraining filament downstream of the treatment site (Block 660).

Once deployed, the delivery catheter is removed from the patient's vascular system and the therapeutic agent(s), if present, is eluted over time (Block 670). Method 600 ends at 680.

Insertion of coated stent 120 into a vessel in the body helps treat, for example, heart disease, various cardiovascular ailments, and other vascular conditions. Catheter deployed coated stent 120 typically is used to treat one or more blockages, occlusions, stenoses, or diseased regions in the coronary artery, femoral artery, peripheral arteries, and other arteries in the body. Treatment of vascular conditions may include the prevention or correction of various ailments and deficiencies associated with the cardiovascular system, the cerebrovascular system, urinogenital systems, biliary conduits, abdominal passageways and other biological vessels within the body.

While the embodiments of the invention disclosed herein are presently considered to be preferred, various changes and modifications can be made without departing from the spirit and scope of the invention. The scope of the invention is indicated in the appended claims, and all changes that come within the meaning and range of equivalents are intended to be embraced therein.

1. A system for treating a vascular condition, the system comprising:
a delivery catheter;
a balloon disposed at a distal end of the delivery catheter;
a stent having a stent framework, the stent disposed on an outer surface of the balloon; and
at least one restraining filament attached to an outer surface of the stent.

2. The system of claim 1 wherein the at least one restraining filament comprises a biodegradable material.

3. The system of claim 1 wherein the at least one restraining filament includes at least one fracture point.

4. The system of claim 1 wherein stent framework comprises a plurality of stent segments and wherein each stent segment includes a restraining filament attached to the stent segment.

5. The system of claim 4 wherein each stent segment includes a filament attachment for attaching a first end of the restraining filament.

6. The system of claim 4 wherein each of the restraining filaments are woven through the stent framework of each stent segment.

7. The system of claim 1 wherein a first end of the restraining filament is secured to a distal end of the stent and wrapped around the stent in a compression configuration and wherein a second end of the restraining filament is secured to a proximal end of the stent.

8. The system of claim 1 further comprising a therapeutic coating having at least one therapeutic agent, the therapeutic coating disposed on at least a portion of an outer surface of the stent.

9. The system of claim 8 wherein the at least one therapeutic agent is selected from a group consisting of anticoagulants, antiinflammatories, fibrinolytics, antiproliferatives, antibiotics, therapeutic proteins, recombinant DNA products, bioactive agents, diagnostic agents, radioactive isotopes, and radiopaque substances.

10. A method of manufacturing a vascular treatment device, the method comprising:
providing a stent, the stent having a stent framework;
securing a first end of a restraining filament to the stent framework;
placing the stent over a balloon disposed at a distal end of a delivery catheter;
compressing the stent onto an outer surface of the balloon;
wrapping the restraining filament around the stent; and
securing a second end of the restraining filament to the stent framework.

11. The method of claim 10 wherein wrapping the restraining filament around the stent further comprises providing a secondary compression to the compressed stent.

12. The method of claim 10 wherein the stent framework comprises a plurality of stent segment, each stent segment having a restraining filament secured to a filament attachment.

13. The method of claim 11 wherein wrapping the restraining filament around the stent comprises weaving the restraining filament through the stent segment to which the restraining filament is secured.

14. The method of claim 10 wherein the provided stent includes a therapeutic coating having at least one therapeutic agent, the therapeutic coating disposed on at least a portion of an outer surface of the stent.

15. The method of claim 14 wherein the at least one therapeutic agent is selected from a group consisting of anticoagulants, antiinflammatories, fibrinolytics, antiproliferatives, antibiotics, therapeutic proteins, recombinant DNA products, bioactive agents, diagnostic agents, radioactive isotopes, and radiopaque substances.

16. A method of treating a vascular condition, the method comprising:
delivering a self-expanding stent including a restraining filament to a treatment site via a balloon catheter, the stent disposed over the balloon;
inflating the balloon to fracture at least one fracture point disposed along the length of the restraining filament;
expanding the self-expanding stent at the treatment site;
contacting an outer surface of the self-expanding stent with a vessel wall at the treatment site; and
trapping the fractured restraining filament between at least a portion of an outer surface of the stent and the vessel wall.

17. The method of claim 16 wherein the self-expanding stent further includes a therapeutic coating on an outer surface, the therapeutic coating including at least one therapeutic agent.

18. The system of claim 17 wherein the at least one therapeutic agent is selected from a group consisting of anticoagulants, antiinflammatories, fibrinolytics, antiproliferatives, antibiotics, therapeutic proteins, recombinant DNA products, bioactive agents, diagnostic agents, radioactive isotopes, and radiopaque substances.

19. The method of claim 17 further comprising eluting at least one therapeutic agent at the treatment site upon contacting the outer surface of the self-expanding stent with the vessel wall at the treatment site.
